(19) United States
(12) Patent Application Publication
(10) Pub. No.: US 2015/0307825 A1
(43) Pub. Date: Oct. 29, 2015

(54) Apparatus and Method for Grain Steeping, Germinating, Kiln Drying and Fermenting, and/or Combinations Thereof

(71) Applicants: Frank-Otto Görlitz, Braunschweig (DE); Kai Silbermann, Braunschweig (DE); Wolf-Eckart Bischoff, Wendeburg (DE)

(72) Inventors: Frank-Otto Görlitz, Braunschweig (DE); Kai Silbermann, Braunschweig (DE); Wolf-Eckart Bischoff, Wendeburg (DE)

(73) Assignee: BUHLER AG, Uzwil (CH)

(21) Appl. No.: 14/348,238

(22) PCT Filed: Sep. 30, 2011
(86) PCT No.: PCT/EP2011/067143

§ 371 Date: Apr. 7, 2015

(51) Int. Cl. C12M 1/16; C12C 1/135; C12M 1/34; C12M 1/06; C12M 1/00

(52) U.S. Cl. CPC C12M 21/16; C12M 27/02; C12M 29/00; C12M 41/00; C12C 1/135

(57) ABSTRACT

The invention relates to a method and a device for steeping, germinating, kilning, fermenting grain, wherein the device comprises a container (3) having at least one plate (20) which can be mounted in the container and having at least one opening (21) for the supply and/or removal of fluid. In addition, the container (3) has at least one base surface (4) wherein, on the base surface (4), substantially vertically arranged side surfaces (5) and, on the base surface (4), substantially vertically arranged end surfaces (9) are present. In addition, a covering surface (6) that is in particular substantially horizontally arranged on at least one side surface (5) and/or end surface (9) is present, as a result of which a process chamber is formed. The internal measurement of a side length of the end surface (9) is 2 to 2.5 meters, in particular 2.345 meters.

The invention relates to an apparatus and to a method for grain steeping, germinating, kiln drying and fermenting, and/or combinations thereof, according to the features of the preambles of the independent patent claims.

Steeping and germinating apparatuses are known in connection with brewing barley, wherein the dormancy of the grains is overcome by increasing the water content during the steeping operation and the brewing barley begins to germinate. The green malt obtained in this connection is subsequently kiln dried or dried and a storable malt obtained in the process. Such apparatuses are referred to as malthouses.

Such steeping and germinating steps are also used in further plant types, such as, together with brewing barley, further types of cereal, pulses, pseudocereals and oil seeds. Within the context of the application, cereals, pseudocereals, oil seeds and pulses and/or constituents thereof and/or mixtures thereof are combined under the term grain.

The prior art discloses malthouses, in which germination is carried out on a plate having openings, also called a perforated plate, for example in a Saladin “germinating box”, by means of temperature controlled and moist air. The fitting of a perforated plate makes it possible to bring water for steeping or air into contact through the perforated plate with the grain to be processed during germinating or kiln drying without the grain to be processed having to be transferred, for example into a further process chamber, between process steps, such as germinating and kiln drying. The water used during steeping becomes soiled during the process and has to be considered to be waste water. For this reason, it is desirable, inter alia, to keep the water consumption low.

DE 101 12 406 discloses an apparatus for steeping and ventilating during the steeping of barley. A perforated plate in this apparatus is configured in such a manner that passage of water into the region, which faces away from the process chamber, under the perforated plate is avoided, and therefore the water consumption can be kept low. The perforated plate is configured merely such that gases can pass from the space below the perforated plate into the process chamber. The realisation of the perforated plate in this apparatus is technically demanding and therefore causes production to be complicated.

WO 2005/083050 discloses an apparatus for steeping barley, wherein under a base having perforations or a perforated plate directly joins passages for conducting water via said passages. By means of this direct feeding of water directly to the perforated plate, the water consumption can be kept low. However, such an apparatus is complicated structurally.

GB 2 407 075 discloses a method for steeping barley by means of three steeping containers. The steeping containers have a perforated plate in the cone, also called conical steeping tank, through which water can be directly supplied and waste water removed. By means of the direct feeding of water, the water consumption can be kept low. A line for the supply of a gas during the steeping operation is arranged above the perforated plate, which is intricate structurally.

It is therefore an object of the present invention to provide an apparatus and a method for steeping, germinating, kiln drying and fermenting, and/or combinations thereof, overcoming the described disadvantages of the prior art. In particular, the intention is to provide a structurally simpler apparatus and a method for steeping, germinating, kiln drying and fermenting, and/or combinations thereof, with reduced water consumption in particular during the steeping of the grain.

According to the invention, this object is achieved by an apparatus and a method having the features of the independent patent claims.

The apparatus according to the invention for steeping, germinating, kiln drying and fermenting, and/or combinations thereof, comprises a container having at least one plate which can be fitted in the container and has at least one opening, in particular for the supply and/or removal of, in particular liquid and/or gaseous, fluid, wherein the container comprises at least one, in particular substantially rectangular, base surface, side surfaces which in particular are arranged substantially vertically, i.e. substantially at an angle of 90°, on the base surface and in particular are parallel to one another, end surfaces which in particular are arranged substantially vertically on the base surface and in particular are parallel to one another, and a top surface which in particular is arranged substantially horizontally on at least one side surface and/or end surface, i.e. substantially parallel to the base surface, for forming a process chamber, and wherein a side length of the end surface corresponds to 2 to 2.5 meters, in particular 2.345 meters, with respect to the interior dimension.

This arrangement corresponds to a structure of an apparatus for grain steeping, germinating, kiln drying and fermenting, and combinations thereof, said structure being easy to realize and being space-saving.

Within the context of the present invention, the side surfaces and/or the end surfaces are the walls, the base surface is the base plate and the top surface is the top plate of the container.

During intended use of the apparatus, a substantially horizontally extending side length of the end surface preferably corresponds to 2 to 2.5 meters, in particular 2.345 meters.

The side surfaces are preferably configured to be longer than the end surfaces. The container therefore in particular advantageously obtains the cuboidal shape of a freight container which is particularly well suited for transportation.

The base surface, side surfaces, end surfaces and the top surface on the side facing the process chamber preferably have a surface and/or coating for foodstuffs. Within the context of the present application, the term suitable for foodstuff is understood as meaning that the grain to be processed is still suitable for human consumption after contact with the surface or coating.

The plate having at least one opening is preferably fittable spaced apart horizontally and substantially parallel to the base surface, in particular on at least one side surface and/or end surface. The plate having at least one opening may be congruent to the base surface and/or top surface. The region above said plate on the side facing away from the base surface substantially forms the process chamber. The plate having at least one opening is preferably in a hinged manner such that access is provided on the side facing away from the process chamber to the space between the base surface and plate in order to facilitate the cleaning of said space. Particularly preferably, the plate is manually pivotable, in particular swingable upward, with respect to an axis of rotation running parallel to a longer side of a side surface and is blockable, in particular supportable, on the opposite side surface. For support purposes, for example, a supporting surface can be provided. In the operating position, the plate is blocked on the opposite side surface.

The plate having at least one opening is preferably arranged substantially parallel to the base surface and/or top surface in the container. A distance (a) of the base surface with respect to the plate having at least one opening to a distance (b) of the plate having at least one opening with respect to a maximum pouring height of the grain is preferably at a length ratio of preferably 1:2 to 1:3, even more preferably of 1:2.25 to 1:2.75, and most preferably of 1:2.4 to 1:2.5.

At such a length ratio of the distance (a) to the distance (b), a simple apparatus is realized, by means of which the water consumption, in particular for steeping the grain, can be kept low at the same time.

Within the context of the invention, a maximum pouring height of the grain is understood as meaning the height to which, during intended use, turning over, i.e. loosening of the grain layer over the entire pouring height, can take place owing to the dimension, i.e. the height, of the turning tools. For example, the maximum pouring height can also be determined from the maximum permissible pressure loss in a flow, which is generated by a device for supplying air, in particular during the germinating, by the pouring height of the grain; for example, the height of the turning tools can be determined from said maximum permissible pressure loss generated by the pouring height of the grain.

The base surface can preferably be configured as a plate having at least one opening. Such an arrangement has the advantage that a particularly large volume is available for receiving grain in the container.

A removal device for removing the grain from the container is arranged, and a turning device which is fittable in the container and in particular is movable and is intended for turning the grain to be processed is contained, preferably in and/or adjacent to the plate having at least one opening. The turning device is furthermore preferably designed as a device for conveying the grain to the removal device, in particular for emptying the container, and therefore the turning device can take on at least a turning-over function for turning purposes, and a clearing-out function for conveying purposes.

Within the context of the present disclosure, a clearing-out function of the turning device is understood as meaning that the turning device is adjustable so as to be used as a device for conveying the grain.

The turning device preferably at least comprises at least one turning screw and/or turning spiral, and a drive for the turning screw or turning spiral.

The at least one turning screw or turning spiral preferably has a complete thread. The turning screw or turning spiral is preferably at a standstill in the clearing-out function and in particular is blockable as a means for conveying the grain.

Within the context of the application, blockable is understood as meaning that the turning screws or turning spirals are controllable so as to substantially prevent a change in the position of the turning screws or turning spirals with respect to one another. This can be undertaken, for example, by controlling and/or regulating the drive.

Furthermore, a planar element can also be arranged at that end of the turning screw or turning spiral which faces the plate, said element, in one position of the turning screws or turning spirals forming a slider for conveying the grain. The positioning of the planar elements to form a slider is preferably adjustable or positionable in a specific manner by means of a position sensor. At least two turning screws or turning spirals having a planar element on the end facing the plate are preferably arranged in the apparatus, said turning screw or turning spirals, in one position, forming a slider for conveying the grain.

The configuration of a turning device for conveying the grain to a removal device serves for a structure of the apparatus according to the invention that is easy to realize and is space-saving.

During intended use, the removal device is preferably arranged fixedly, i.e. nonmovably, in the apparatus. This advantageously simplifies the structure of the apparatus according to the invention.

Furthermore, the removal device is preferably fitted in the process chamber in particular adjacent to a side surface and/or end surface. By means of this arrangement, the conveying of the grain towards the removal device is simplified. Furthermore, the removal device is preferably arranged in the space between the base surface and the plate having at least one opening.

The removal device preferably comprises at least one removal container tapering conically in sections in the conveying direction, i.e. in the direction of the removal device, and/or a screw conveyor, in particular arranged in the conically tapering removal container.

Furthermore, the conically tapering removal container is preferably formed in a complementary manner with respect to the cross section of the screw conveyor, i.e. at the position of the screw conveyor, the removal container is formed in a complementary manner with respect to the round cross section of said screw conveyor. As complete an emptying as possible of the grain to be processed from the container is therefore facilitated.

Furthermore, an, in particular closable, clearing-out flap and/or a flat slider may be fittable on the removal device in order to open up or to block access of the grain to/from the removal container. By the fitting of a clearing-out flap or of a flat slider, a barrier blocking the removal container in order to prevent the grain to be processed from exiting during the process steps is advantageously formed, said barrier being able to be opened up in order to empty the grain via the removal container. The clearing-out flap and/or the flat slider may be operable manually, pneumatically or hydraulically. The clearing-out flap is preferably hinged in the direction of the conically tapering removal container. The flat slider is preferably in particular manually operable by means of a device on a container outer surface facing away from the process chamber.

Within the context of the invention, conically is understood as meaning a narrowing cross sectional area substantially perpendicular to the inlet opening for grain into the removal container.

An angle α which is opened at least in sections between a surface formed by an inlet opening in the removal device and the conical supply direction to the removal container lies within the range of 10 to 80°, preferably of 30 to 70°, even more preferably of 50 to 60°.

The angle α is therefore selected in such a manner that the distance a between base surface and plate having at least one opening is firstly as small as possible and secondly a very substantial conveying of the grain to the removal device, in particular to the screw conveyor, by means of the turning device is ensured, in particular for the very substantial emptying of the processed grain from the container, and a screw conveyor ensures a high discharge capacity by means of as large a cross section as possible.

Furthermore, the turning device is preferably movable, in particular displaceable, toward the removal device. This has the advantage that the grain can be emptied as substantially as possible from the container using the removal device.

During intended use, the side surfaces and/or end surfaces are preferably arranged fixedly, i.e. non-displaceably, on the base surface, thus ensuring the required stability of the container during transportation.

During intended use, the turning screws or turning spirals are preferably designed to be height adjustable substantially parallel to and/or antiparallel to the direction of gravity. Furthermore, the turning screws or turning spirals may be pivotable with respect to an axis of rotation running parallel substantially with respect to the end wall, in particular from a vertical position, running substantially parallel to gravity, into a horizontal position.

This advantageously permits manipulations to the turning tools to be undertaken in a simple manner.

The turning device preferably comprises at least one guide which is fitted on at least one, in particular reinforced, side surface and/or end surface, and a drive for displacing the turning device along the guide.

The guide is preferably configured as a strip having a rack and pinion gearing. Furthermore, the turning device may be displaceable along the strip by means of a guide roller, wherein the guide roller is frictionally connected by means of a shaft, in particular a force transmission shaft, to the drive for displacing the turning device.

At least one, preferably two, side surface(s) and/or end surface(s) of the container preferably has/have at least one, in particularly substantially complementary, recess at least for the partial receiving of the turning device, in particular the turning screws or turning spirals.

Such a recess has the advantage that, when the removal device is fitted adjacent to a side surface and/or end surface, the turning device can be displaced to a position close to the removal device in order to obtain very substantial emptying of the processed grain from the container without a large portion of the grain being conveyed by the turning device over the removal device and subsequently no longer being conveyable by the turning device into the removal device, as may be the case, for example, when adjacently arranged turning spirals are used.

An, in particular bendable, energy guiding device, in particular a cable duct or an energy guiding chain, is preferably arranged on a side surface and/or end surface and is operatively connected to the turning device, wherein, in particular, cables for supplying energy to the moveable turning device can be accommodated in the energy guiding device.

During intended use, the energy guiding device is preferably bendable in a plane substantially parallel to the top surface.

The energy guiding device is preferably bendable along two substantially parallel guide rails, wherein the guide rails extend in particular substantially parallel to the side surfaces. The guide rails are preferably arranged spaced apart from each other substantially in a plane parallel to the top surface. The guide rails are preferably arranged in a space between the maximum pouring height and the top surface. The second guide rail is preferably arranged on a side surface and/or top surface.

The first guide rail preferably extends substantially along the length of the side surfaces. The second guide rail preferably has substantially a length within the range of 40% to 60% of the length of the first guide rail and extends in particular from an end surface as far as the center of a longer side of a side surface.

Furthermore, the energy guiding device, in particular at the ends, preferably has at least two fixing means in the container, wherein one fixing means is fitted on the turning device and a further fixing means is fitted, in particular in the region of the center of a longer side of the side surface, on the top surface, the side surface or the second guide rail.

This manner of arranging the energy guiding device has the advantage that there is hardly any reduction in the grain-receiving volume between the plate having at least one opening and the top surface.

A wetting device is preferably finable on the turning device. The grain is preferably wettable with water. In a particularly preferred manner, additives, in particular dissolved in water, can be fed into the container.

This has the advantage that water or, for example, vitamins and/or minerals can be fed in in accordance with the process requirements in order to increase the quality of the final product.

Furthermore, the wetting device may preferably be configured as a cleaning device which in particular facilitates cleaning-in-place cleaning. A cleaning device is particularly preferably formed as a separate device, in particular in addition to the wetting device.

There is preferably at least one opening, particularly preferably a plurality of openings, in particular along an axis on a distributing device, for feeding grain into the container through the at least one opening.

Furthermore, a distributing device is understood as meaning a device for the distributed supply of the grain to be processed into the container at at least one position. By means of the distributed supply of the grain to be processed through various openings, an outlay on distributing the grain within the process chamber is advantageously reduced.

The distributing device is preferably fitted substantially along an axis, in particular on the top surface, which runs substantially parallel to the side surfaces of the container. By fitting the distributing device on the top surface, the grain to be processed can pass by means of the effect of gravity into the process chamber without conveyor means additionally being required.

The distributing device preferably comprises at least one conveyor device in order to convey the grain to be processed to the desired position. The conveyor device facilitates controlled conveying of the grain to be processed.

The distributing device and/or the conveyor device is preferably able to be controlled and/or regulated.

In particular, the distributing device comprises blocking sliders which can be regulated and/or controlled, in particular clocked, for opening and/or closing the openings. This has the advantage of a further reduction in the outlay on distributing the grain within the process chamber and of more uniformly distributing the grain.

The apparatus preferably comprises at least one device for the supply of an, in particular conditioned, fluid, in particular for water for steeping the grain and/or for a gas.

The device for the supply of water may be configured as a separate device from the device for the supply of a gas. The term gas is understood as meaning, for example, conditioned air for the process steps of germinating and kiln drying. The gas or the conditioned air is particularly preferably supplied by means of a fluid line below the plate having at least one opening.

Preferably, the base surface, side surfaces, end surfaces and/or top surface of the container, or all possible combinations thereof, are double-walled, for example the following combinations: base surface, side surfaces and end surfaces; top surface, side surfaces, end surfaces; base surface and side surfaces; base surfaces and end surfaces; side surfaces and end surfaces; base surface and top surface; side surfaces and top surfaces; end surfaces and top surface, are preferably double-walled. Within the context of the invention, the term double-walled should be understood as meaning that the walls of the surfaces are at least partially spaced apart from one another.

This has the advantage that the container is better insulated in particular in relation to external temperature influences and in particular in relation to temperature fluctuations, wherein the container is insulated in relation to cold during the germinating operation and in relation to heat losses during the kiln drying operation. An additional insulating layer may optionally be present in the space in between the two walls.

As a further advantage, a double-walled container provides improved protection of the container interior in relation to damage, for example during transportation of the apparatus.

The apparatus is preferably configured in two parts, wherein a first apparatus element comprises at least the container, and a second apparatus element comprises at least one device for the supply of a gas, in particular conditioned air, into the container. By division of the apparatus, the transportation for the two respective elements is simplified.

A further aspect of the invention relates to a method, which is carried out in particular by the apparatus according to the invention, for treating grain, in particular cereals, pseudocereals, oil seeds or pulses. First of all, a container is filled with water and grain. In a subsequent process step, the grain is steeped by the water. After the end of the steeping step, the water is emptied from the container. Subsequently, in a next process step, the grain is germinated, in particular with the supply and/or removal of conditioned air, and/or in particular turning the grain by means of a turning device. Subsequently, optionally as a further process step, the grain is kiln dried, for example to a water content of 2 to 14% by weight. As a further optional process step, in particular after germination, the grain can be fermented by hermetic sealing in relation to the surroundings. The process steps of kiln drying and fermenting are interchangeable in the sequence thereof.

A further aspect of the invention relates to the use of the device described above for grain steeping, germinating, kiln drying and fermenting, and/or combinations thereof, in particular as claimed in the method described above, wherein the apparatus is configured in particular to be mobile.

Within the context of the invention, the term mobile is understood as meaning that the apparatus according to the invention is transportable by boat, truck and comparable transportation means. The apparatus according to the invention can preferably be fittable in an ISO freight container. The first apparatus element is particularly preferably a 40 foot ISO freight container (internal length: 12.040 meters; internal width: 2.345 meters; internal height: 2.385 meters) or a 40 foot high cube ISO freight container (internal height: 2.690 meters in contrast to the 40 foot ISO freight container), and the second apparatus element is particularly preferably a 20 foot ISO freight container (internal length: 5.910 meters; internal width: 2.345 meters; internal height: 2.385 meters).

A further aspect of the invention relates to a unit which may comprise at least one device for the supply and/or removal of an, in particular conditioned, fluid, in particular for water for steeping the grain and/or for a gas.

Conditioning the fluid is understood as meaning in particular cooling the gas, in particular during germination, or heating the gas, in particular during the kiln drying operation. The unit preferably has a temperature control device for the fluid.

Furthermore, the unit may comprise only supply lines and/or removal lines for the fluid and may be fittable to a further supply device having a device for the supply and/or removal of a fluid.

In particular, such a unit is suitable for receiving an apparatus in which the plate having at least one opening is configured as the base surface, in order to form an apparatus for grain steeping, germinating, kiln drying and fermenting, and combinations thereof.

The unit may be installable fixedly and/or configured to be mobile.

A further aspect of the invention relates to a kit comprising a unit having at least one device for the supply and/or removal of a fluid, and the apparatus, in which the plate is configured as the base surface.

The configuration of a container with a plate having at least one opening as the base surface has the advantage that the receiving volume in the case of, for example, predetermined exterior dimensions, such as in the case of a 40 foot ISO freight container, can be maximized. In addition, an apparatus of this type is more simply transportable, since the unit does not necessarily have to be transported therewith. For example, units may be installed in each case at two locations, and therefore, depending on the particular requirement, only the apparatus has to be transported to the particular location. In particular, an apparatus with a plate as the base surface is placed onto the unit, as a result of which the unit has a sealing effect toward the surroundings in particular at the location of the baseplate as a plate having at least one opening in the apparatus.

A further aspect of the invention relates to a method for the operationally ready installation of an apparatus for grain steeping, germinating, kiln drying and fermenting, and combinations thereof, wherein the apparatus with a plate having at least one opening as the base surface of a container and the unit are functionally connected to each other.

The invention is explained in more detail below with reference to the drawings without restricting the invention thereto. In the drawings:

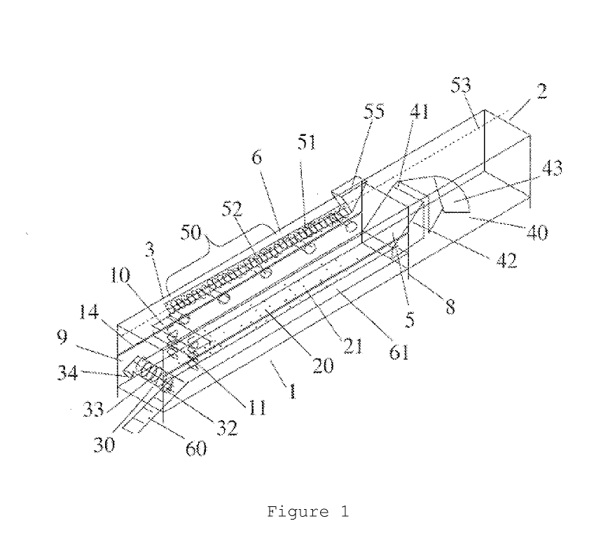

FIG. 1 shows a perspective illustration of a first variant embodiment of the apparatus according to the invention with a footbridge in the first apparatus element.

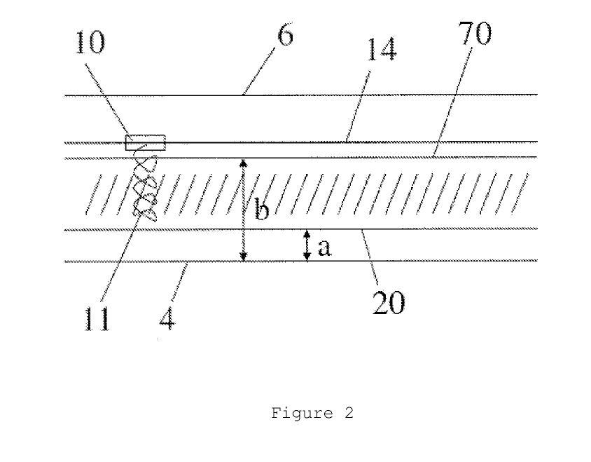

FIG. 2 shows a sketched side view of an apparatus according to the invention

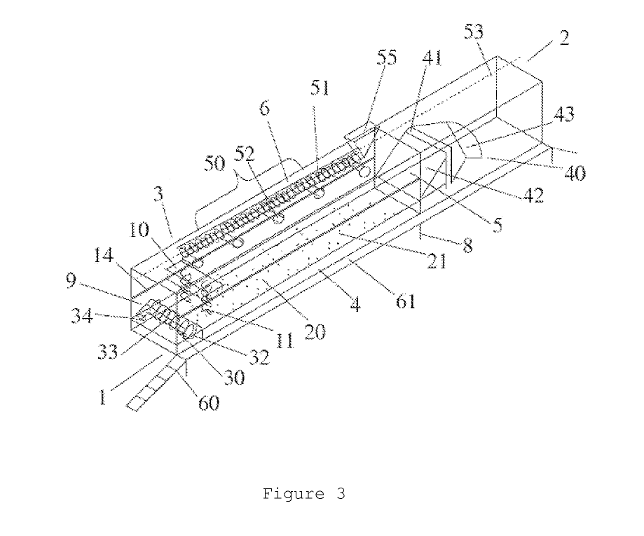

FIG. 3 shows a perspective illustration of a second variant embodiment of the apparatus according to the invention with a footbridge outside the first apparatus element

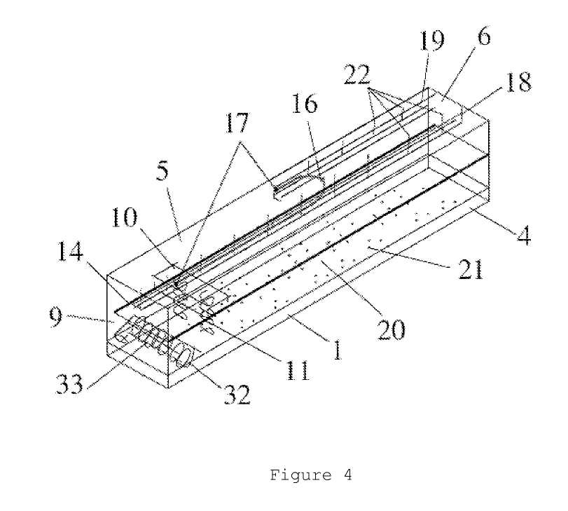

FIG. 4 shows a perspective illustration of the apparatus according to the invention with an energy chain guide having two guide rails, which are arranged horizontally with respect to each other, for the energy guiding device

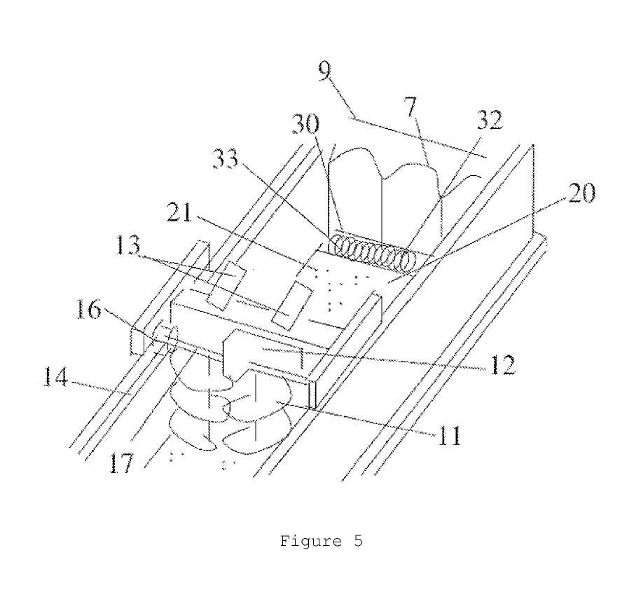

FIG. 5 shows a perspective illustration of the turning device of the apparatus according to the invention under the top surface.

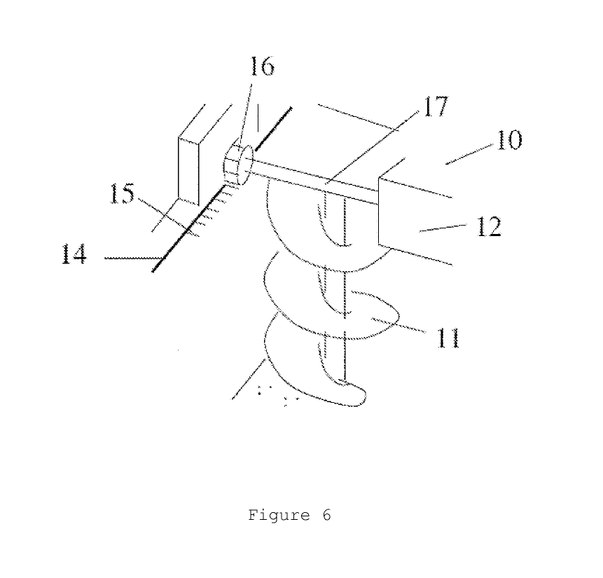

FIG. 6 shows a perspective illustration of the positioning of the turning device in the apparatus according to the invention

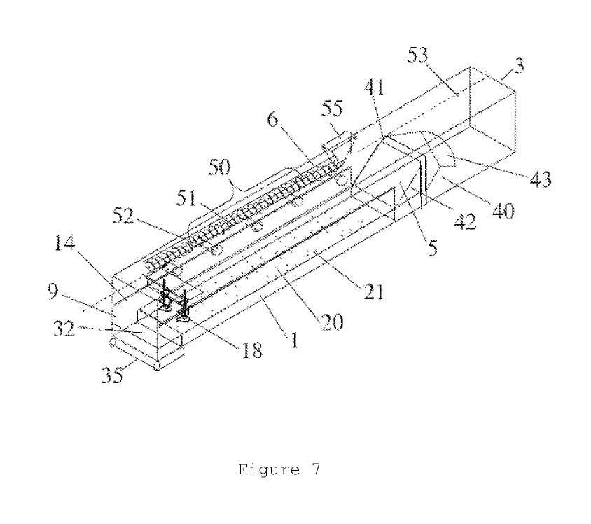

FIG. 7 shows a perspective illustration of a third variant embodiment of an apparatus according to the invention configured as a single part

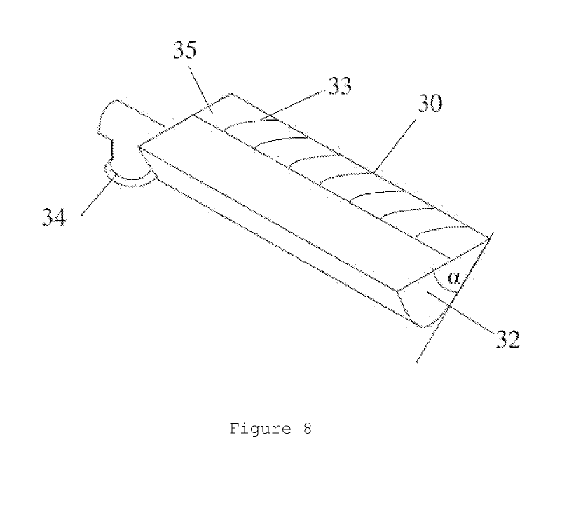

FIG. 8 shows a schematic illustration of a removal device of the apparatus according to the invention

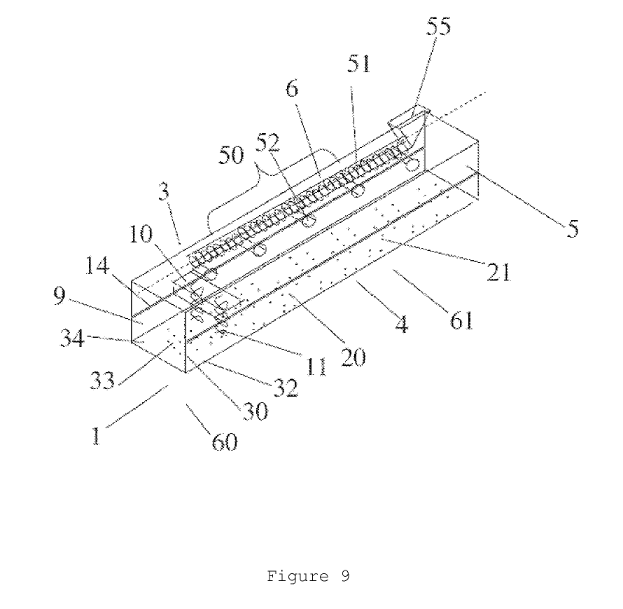

FIG. 9 shows a perspective illustration of a fourth variant embodiment of an apparatus according to the invention with a configuration of the plate as base surface

FIG. 1 shows a first variant embodiment of the apparatus according to the invention in a perspective illustration. FIG. 1 shows a first apparatus element 1 which comprises a container 3 with a base surface 4, two parallel side surfaces 5, two parallel end surfaces 9 and a top surface 6. Furthermore, FIG. 1 shows a turning device 10 with two turning screws 11, which is arranged displaceably on a strip 14 in the container 3. Furthermore, in FIG. 1, a plate 20 having openings 21 is fitted on the side surfaces 5 and end surfaces 9 in a manner spaced apart horizontally and parallel to the base surface 4. FIG. 1 shows a distributing device 50 on the top surface 6 of the container 3 for the supply of the grain to be processed along an axis 53, said distributing device comprising a charging hopper 55, a screw conveyor 51 as the conveyor device and five openings 52. A removal device 30 with a conically tapering removal container 32 and a screw conveyor 33 arranged in the conically tapering removal container, and also a removal opening 34 furthermore showing in FIG. 1. FIG. 1 shows a footbridge 61 in the container 3, which can be accessed by means of a staircase 60 and a door (not visible in FIG. 1) on the end surface 9. In a second apparatus element 2 of this variant embodiment of the apparatus according to the invention as per FIG. 1, a device 40 for the supply of a gas is shown, said device comprises a ventilator 43, a heat exchanger 41 and a plurality of directional baffles 42 for guiding the air through an opening (not visible in FIG. 1) between the first apparatus element (1) and second apparatus element (2) under the plate 20. Furthermore, FIG. 1 shows a basic framework 8 on which the first apparatus element (1) and the second apparatus element (2) are fitted.

From here onward and below, the same reference numbers refer to identical components in the figures.

FIG. 2 shows a sketched side view of an apparatus according to the invention, wherein the distance a between the base surface 4 of the container 3 and the plate 20 and the distance b between the plate 20 and the maximum pouring height 70 are visible. In a preferred embodiment of the apparatus according to the invention, the distance a corresponds to 465 millimeters and the distance b to 1,150 millimeters.

FIG. 3 shows a second variant embodiment of the apparatus according to the invention in a perspective illustration. By contrast to FIG. 1, FIG. 2 additionally shows a footbridge 61 which is fitted on the basic framework 8 outside the container 3.

FIG. 4 shows a perspective illustration of the apparatus according to the invention with an energy guiding device 16 between a first guide rail 18 and a second guide rail 19, which are arranged substantially parallel to each other and are substantially spaced apart from each other merely horizontally, i.e. are arranged on a level. As is apparent in FIG. 4, the two guide rails are fastened to the top surface 6 by means of struts 22. FIG. 4 furthermore shows a fixing means 17 of the energy guiding chain 16 to the second guide rail 19 and a further fixing means 17 of the energy guiding chain 16 to the turning device 10. In addition, FIG. 4 shows that the first guide rail extends over the length of the strip 14 as a guide of the turning device 10.

FIG. 5 shows a perspective illustration of a turning device 10 of the second variant embodiment of the apparatus according to the invention as per FIG. 2 below the top surface (not visible in FIG. 3). FIG. 5 shows a drive 12 for displacing the turning device 10 by means of a shaft 17 and a guide roller 16 along the strip 14. FIG. 5 furthermore shows two drives 13 of the turning screws 11. In addition, FIG. 5 shows, adjacent to the plate 20 having openings 21, the removal device 30, comprising the removal container 32 and the screw conveyor 33. FIG. 5 furthermore shows a complementary recess 7 to the turning screws 11 on the end surface 9 of the container 3.

FIG. 6 shows a perspective illustration of the positioning of the turning device 10 on the container 3 of the apparatus according to the invention. In addition to FIG. 5, a lantern tooth system 15 on the strip 14 is shown interacting with the guide roller 16, which is configured as a toothed wheel, in FIG. 6.

FIG. 7 shows a perspective illustration of a single-part apparatus according to the invention, wherein the container 3 additionally comprises the device 40 for the supply of a gas, with the ventilator 43, the heat exchanger 41 and a plurality of directional baffles 42 for guiding the air through an opening below the plate 20. In the container 3 according to FIG. 7, recesses according to FIG. 5 on the end surfaces are omitted. FIG. 7 also shows the turning device 10 having two turning spirals 18, which is arranged displaceably on the strip 14 in the container 3. Furthermore, FIG. 7 shows a removal device 30 with a removal container 32 which does not taper conically and in which a conveyor belt 35 is fitted. FIG. 7 also shows the distributing device 50 for the supply of grain to be processed along the axis 53 on the top surface 6 of the container 3, said distributing device comprising the collecting hopper 55, a screw conveyor 56 as the conveyor device and five openings 52.

FIG. 8 shows a schematic illustration of the removal device 30 of an apparatus according to the invention with a removal container 32, screw conveyor 33 and the inlet opening 35. Furthermore, an angle α which is opened between a surface formed by the inlet opening 35 in the removal device 30 and the conical supply direction to the removal container 32 is visible.

FIG. 9 shows a perspective illustration of an apparatus according to the invention, wherein the plate 20 having at least one opening 21 is configured as the base surface 4 of the container 3.

1-21. (canceled)

22. An apparatus for grain steeping, germinating, kiln drying and fermenting, or combinations thereof, wherein the apparatus comprises a container having at least one plate which can be fitted in the container and has at least one opening, wherein the container comprises at least one base surface, side surfaces, end surfaces, and a top surface, for forming a process chamber, and wherein a side length of the end surface corresponds to 2 to 2.5 meters with respect to the interior dimension.

23. The apparatus as claimed in claim 22, wherein the plate has at least one opening and is arranged substantially parallel to the base surface and/or top surface in the container, and a length ratio of a distance of the base surface with respect to the plate having at least one opening with respect to a distance of the plate having at least one opening to a maximum pouring height of the grain is 1:2 to 1:3.

24. The apparatus as claimed in claim 22, wherein the base surface is configured as a plate having at least one opening.

25. The apparatus as claimed in claim 22, wherein a removal device for removing the grain from the container is arranged, and a turning device which is fittable in the container and is intended for turning the grain to be processed is contained, in and/or adjacent to the plate having at least one opening, wherein the turning device is designed as a device for conveying the grain to the removal device.

26. The apparatus as claimed in claim 25, wherein the turning device comprises at least one turning screw and/or turning spiral, and a drive for the turning screw and/or turning spiral.

27. The apparatus as claimed in claim 25, wherein the removal device comprises at least one removal container tapering conically in sections in the conveying direction and/or a screw conveyor.

28. The apparatus as claimed in claim 27, wherein an angle a which is opened at least in sections between a surface formed by an inlet opening in the removal device and the conical supply direction to the removal.

29. The apparatus as claimed in claim 25, wherein the turning device is movable toward the removal device.

30. The apparatus as claimed in claim 22, wherein, during intended use, the side surfaces and/or end surfaces are arranged fixedly on the base surface.

31. The apparatus as claimed in claim 25, wherein the turning device comprises at least one guide which is fitted on at least one side surface and/or end surface, and a drive for displacing the turning device along the guide.

32. The apparatus as claimed in claim 25, wherein at least one side surface and/or end surface of the container has at least one recess at least for the partial receiving of the turning device.

33. The apparatus as claimed in claim 22, wherein there is at least one opening for feeding grain into the container through the at least one opening.

34. The apparatus as claimed in claim 33, wherein the distributing device comprises at least one conveyor device.

35. The apparatus as claimed in claim 33, wherein the distributing device and/or the conveyor device is/are controllable and/or regulatable.

36. The apparatus as claimed in claim 35, wherein the apparatus comprises at least one device for the supply of a fluid.

37. The apparatus as claimed in claim 36, wherein the apparatus is configured in two parts, wherein a first apparatus element comprises at least the container and a second apparatus element comprises at least one device for the supply of a gas into the container.

38. A method for treating grain, in particular cereals, pseudocereals, oilseeds or pulses, containing at least the following process steps:
(i) filling a container with water and grain,
(ii) steeping the grain in the water,
(iii) germinating the grain,
(iv) optionally kiln drying the grain,
(v) optionally fermenting the grain, and
(vi) emptying the grain treated in the previous steps from the container,
wherein the method is carried out in an apparatus as claimed in claim 22.

39. A unit suitable for receiving an apparatus as claimed in claim 24, the unit comprising at least one device for the supply and/or removal of a fluid.

40. A kit comprising at least one apparatus as claimed in claim 24 and a unit for receiving the apparatus, said unit comprising at least one device for the supply and/or removal of a fluid.

41. A method for the operationally ready installation of an apparatus for grain steeping, germinating, kiln drying and fermenting, and combinations thereof, wherein an apparatus as claimed in claim 24 and a unit for receiving the apparatus, said unit comprising at least one device for the supply and/or removal of fluid, are functionally connected to each other.
